(19) United States
(12) Patent Application Publication
(10) Pub. No.: US 2023/0380829 A1
(43) Pub. Date: Nov. 30, 2023

(54) SUTURE-ANCHOR INSERTION DEVICE

(71) Applicant: OSSIO LTD, Caesarea (IL)

(72) Inventors: Tal ZEEVI, Pardes Hana-Karkur (IL); Ido ASHPIZ, Rosh Haayin (IL); Orahn PREISS-BLOOM, Zichron Yaakov (IL)

(21) Appl. No.: 18/193,265

(22) Filed: Mar. 30, 2023

Related U.S. Application Data
(60) Provisional application No. 63/347,021, filed on May 30, 2022.

(51) Int. Cl. A61B 17/04

(52) U.S. Cl. CPC A61B 17/0401; A61F 2002/0852

(57) ABSTRACT

The present invention is directed to a suture-anchor insertion device used to insert a suture anchor into bone, such that the suture anchor secures soft tissue to the bone in a manner that does not require the surgeon to tie suture knots to secure the tissue to the bone. The apparatus for securing a suture to a bone, comprises a suture anchor configured to be inserted into a hole in the bone, the suture anchor comprises: a tip component configured to be advanced into the hole, the tip component comprises: a widened end portion that defines an eyelet that is configured to receive the suture; a shaft that projects axially from the widened end portion; and a cannulated screw configured to secure the tip component within the hole within the bone by being advanced over the shaft of the tip component; and a suture-anchor insertion device comprises: a handle portion; one or more rods; and one or more spools configured to releasably secure proximal ends of the suture by the proximal ends of the suture being wound around the one or more spools.

### FIELD OF EMBODIMENTS OF THE INVENTION

The present invention relates to medical apparatus and methods, and specifically to apparatus and methods for securing soft tissue to bone.

### BACKGROUND

Grafts are typically secured to bone via tissue-affixing sutures. Using traditional methods, the tissue-affixing sutures are knotted by the surgeon performing the procedure. More recently, suture anchor devices have been developed for securing soft tissue to bone in a manner that does not require the surgeon to tie suture knots to secure the tissue to the bone. Some such devices include a tip component that defines an eyelet for holding one or more tissue-affixing sutures within a pilot hole. The tissue-affixing suture(s) are secured into the hole by inserting the eyelet into bone and advancing a plug, such as a cannulated screw, over a shaft of the tip component.

### SUMMARY OF EMBODIMENTS

In accordance with some applications of the present invention, a suture-anchor insertion device is used to insert a suture anchor into bone, such that the suture anchor secures soft tissue to the bone in a manner that does not require the surgeon to tie suture knots to secure the tissue to the bone. Typically, the suture anchor includes a tip component that defines an eyelet. The tip component is configured to hold one or more sutures (e.g., between one and four sutures), which are typically tissue-affixing sutures, within a pilot hole in the bone. For some applications, some or all of the tissue-affixing sutures are attached to a curved or straight needle at one end or at both ends. The tissue-affixing sutures are typically threaded through one or more tendons or ligaments and are used to approximate the tendons or ligaments to the bone surface and to affix the tendons or ligaments to the bone.

For some applications, the tip component defines a widened end portion toward a first (distal) end of the tip component, which is configured to be advanced to the bottom of the pilot hole. The widened end portion typically defines the eyelet, through which the sutures are threaded. Typically, the tip component includes a shaft, which extends axially from the widened end portion of the tip component toward a second (proximal) end of the tip component (which is configured to be placed closer to the top of the pilot hole). (The term “proximal” is used herein to denote a portion that is closer to a user of the device (e.g., an orthopedic surgeon), while the term “distal” is used to denote a portion that is farther from the user.)

For some applications, the suture anchor includes a plug component, which is configured to secure the tip component within the pilot hole by being advanced into and thereby plugging the pilot hole. Typically, the plug component is a cannulated screw, which is configured to secure the tip component within the pilot hole by being screwed into the pilot hole to thereby plug the pilot hole. The cannulated screw typically defines a lumen therethrough, and the cannulated screw is typically advanced over the shaft of the tip component, such that the shaft is disposed within the lumen of the cannulated screw.

The suture-anchor insertion device typically includes a handle portion that includes a proximal rotation knob and a distal pusher. Typically, a pushing rod extends distally from the distal pusher to the proximal end of the cannulated screw. Further typically, a rotation rod extends distally from the proximal rotation knob to the cannulated screw and through the lumen of the cannulated screw. For some applications, the rotation rod extends through the inside of the pushing rod. For some applications, the cannulated screw is advanced into the pilot hole and over the shaft of the tip component by using the pushing rod to push the screw distally, while simultaneously using the rotation rod to rotate the screw, and thereby screw the screw into the bone.

In accordance with some applications of the present invention, proximal ends of the tissue-affixing suture(s) are secured by being wound around spools. Typically, the proximal ends of the tissue-affixing suture(s) are wound around the spools, such as to prevent damage to the tissue-affixing suture(s) (e.g., twisting and/or knotting of the tissue-affixing suture(s)) while the tissue-affixing suture(s) are disposed within the packaging. Typically, the spools secure the proximal ends of the tissue-affixing suture(s) with respect to the pushing rod and/or with respect to handle portion. For some applications, prior to insertion of the suture anchor and/or during the insertion of the suture anchor, the user is able to adjust the tension of the tissue-affixing suture(s) by winding the spools. For some applications, some or all of the tissue-affixing sutures are attached to a curved or straight needle at one end or at both ends. For some applications, the spools are configured to safely store the needles. For example, the spools may define holes that are shaped so as to accommodate receive tips of the needles and to hold the needles in a way that does not expose the sharp tips of the needles. For some applications, the needles are curved and the radius of curvature of spool is similar to that of the needles, such that the spool accommodates the curvature of the needle.

Typically, after suture anchor has been inserted (an in some cases prior to the suture anchor having been inserted), the tissue-affixing sutures are released from the spools by pulling the tissue-affixing sutures while allowing the spools to rotate and release the strands, for example by holding the sides of the spools lightly and allowing them to rotate. For some applications, the spools define round indents on their outer surfaces such as to allow a user to insert their fingers into the indents and thereby hold the spools while they are rotated as the sutures are pulled off. Typically, the spools are detachably attached to the suture-anchor insertion device such that the spools are configured to remain attached while the suture anchor is being inserted but can be readily removed following suture anchor insertion. For some applications, the user removes the spools from the suture-anchor insertion device prior to the insertion of the suture anchor. For some applications, the spools are attached to pushing rod of the suture-anchor insertion device. For some such applications, the pushing rod includes a segment having a non-smooth outer surface (e.g., a segment that defines teeth, waves, and/or a ribbed segment) in order to facilitate coupling of the spools to the pushing rod. Typically, the suture-anchor insertion device includes two spools (e.g., with one spool on each side of the suture anchor). For some such applications, the spools are coupled to each other. Typically, the spools are removable from the pushing rod with a single motion, for example by pulling the spools from the pushing rod. Typically, the spool is comprised of a material having the same or higher flexibility than the flexibility of the pushing rod.

For some applications, the handle portion of the insertion device defines shoulder cleats. For example, the distal end of the handle portion (i.e., the distal end of the pusher) may define shoulder cleats. Typically, the shoulder cleats extend substantially laterally with respect to the handle portion. (It is noted that the shoulder cleats may also extend in additional directions, other than the lateral direction. For example, the shoulder cleats may be shaped to define a proximally-facing concave curvature.) For some applications, the shoulder cleats extend laterally to the edges of the handle portion. Alternatively, the shoulder cleats extend laterally past the lateral edges of the handle portion. For some applications, as an alternative to or in addition to winding the proximal ends of the tissue-affixing suture(s) around spools, the proximal ends of the tissue-affixing suture(s) are inserted into the shoulder cleats. Typically, the proximal ends of the tissue-affixing suture(s) are secured by the shoulder cleats, such as to prevent damage to the tissue-affixing suture(s) (e.g., twisting and/or knotting of the tissue-affixing suture(s)).

Typically, the shoulder cleats include a trapping mechanism (e.g., a male-female interlocking structure) for mechanically trapping the proximal ends of the tissue-affixing suture(s) such that the proximal ends of the tissue-affixing suture(s) cannot be pulled out longitudinally but can be removed by pulling the proximal ends of the tissue-affixing suture(s) laterally. As described hereinabove, for some applications, the shoulder cleats extend laterally outwardly past the lateral edges of the handle portion. For some applications, the suture-anchor insertion device is provided to a user without the tissue-affixing suture(s) having been preloaded, but the insertion device is provided with shoulder cleats. Typically, once the tissue-affixing suture(s) has been inserted through the eyelet of the tip component by the user, the user then inserts the proximal ends of the tissue-affixing suture(s) into the shoulder cleats. For some such applications, the lateral extensions of the shoulder cleats (which extend laterally past the lateral edges of the handle portion), allow the user to initially adjust the tension of the tissue-affixing suture(s) by pulling proximal ends of the tissue-affixing suture(s) around the lateral extensions. Subsequently, the tissue-affixing suture(s) are secured at the desired tightness by sliding the proximal ends of the tissue-affixing suture(s) laterally into the trapping mechanism.

For some applications, the suture-anchor insertion device includes a stay suture and a stay-suture release tab. The stay suture typically passes through a stay-suture eyelet defined by the shaft of the tip component of the suture anchor, and then passes through a lumen defined by the rotation rod to the handle portion. The stay suture is typically provided preloaded within the insertion device such that the tip component is fixed to the distal end of the rotation rod. At its proximal end, the stay suture is held in position with respect to the rotation knob via the stay-suture release tab. In order to release the tip component from the insertion device once the suture anchor has been properly implanted, the stay-suture release tab is typically manually removed (e.g., pulled away and/or ripped). For some applications, the stay-suture release tab is made of a manually tearable material, such as paper or fabric, that is configured to be manually torn such as to release the stay suture. Alternatively, the stay-suture release tab is made of is made of a non-tearable material (e.g., a material made of high-density spunbound polyethylene fibers, such as Tvvek®) that is configured to be pulled away such as to release the stay suture. In an alternative embodiment, a mechanical element such as a button or a lever is used to release the stay suture. Further alternatively, the stay suture may be released by opening up the handle portion (e.g., by opening up the rotation knob of the handle portion).

There is therefore provided, in accordance with some applications of the present invention, apparatus for securing a suture to a bone, the apparatus including:
a suture anchor configured to be inserted into a hole in the bone, the suture anchor including:
   a tip component configured to be advanced into the hole, the tip component including:
      a widened end portion that defines an eyelet that is configured to receive the suture;
      a shaft that projects axially from the widened end portion; and
   a cannulated screw configured to secure the tip component within the hole within the bone by being advanced over the shaft of the tip component; and
a suture-anchor insertion device including:
   a handle portion;
   one or more rods; and
   one or more spools configured to releasably secure proximal ends of the suture by the proximal ends of the suture being wound around the one or more spools.

In some applications, the one or more spools are configured to secure the proximal ends of the suture, such as to prevent twisting and/or knotting of the suture.

In some applications, the one or more spools are configured to secure the proximal ends of the suture, such as to hold the proximal ends of the suture out of the way of the suture-anchor insertion device.

In some applications, the apparatus includes the suture, and the suture anchor insertion device is provided to a user with the suture passing through the eyelet and with proximal ends of the suture wound around the one or more spools.

In some applications, the suture anchor insertion device is provided to a user without the suture passing through the eyelet and the apparatus is configured for a user to insert the suture through the eyelet and to wind the proximal ends of the suture around the one or more spools.

In some applications, the one or more spools are configured such that the proximal ends of the sutures are configured to be released from the one or more spools prior to advancing the suture anchor into the hole in the bone.

In some applications, the one or more spools are configured such that the proximal ends of the sutures are configured to be released from the one or more spools subsequent to advancing the suture anchor into the hole in the bone.

In some applications, the one or more spools are configured to allow a user to adjust tension of the suture by winding the one or more spools.

In some applications, the suture anchor defines a stay suture eyelet, and the apparatus further includes:
a stay suture configured to pass through the stay suture eyelet and into the handle portion, by passing along a lumen defined by one of the one or more rods; and
a stay-suture release tab configured to releasably hold a proximal end of the stay suture within the handle portion.

In some applications, the apparatus is configured for use with a suture that is coupled to a needle, and the one or more spools are configured to accommodate the needle.

In some applications, the apparatus is configured for use with a suture that is coupled to a curved needle, and the one or more spools has a curvature that is such as to accommodate a curvature of the curved needle.

In some applications, the one or more spools define one or more holes that are configured to receive a tip of the needle.

In some applications, the one or more spools are detachably coupled to the one or more rods.

In some applications, the one or more spools are configured to be detached from the one or more rods prior to advancing the suture anchor into the hole in the bone.

In some applications, the one or more spools are configured to be detached from the one or more rods subsequent to advancing the suture anchor into the hole in the bone.

In some applications, the one or more rods include a segment having a non-smooth surface to facilitate coupling of the one or more spools to the one or more rods.

In some applications, the one or more spools includes two or more spools that are coupled to each other, such as to be detached from the one or more rods simultaneously with each other.

In some applications, the two or more spools are configured to be detached from the one or more rods with a single motion.

In some applications, one or more spools are configured such that the suture is released from the spools by pulling the sutures while allowing the spools to rotate and release the suture.

In some applications, the one or more spools define round indents on outer surfaces thereof, which are configured such as to allow a user to insert their fingers into the indents and thereby hold the spools while the spools are rotated.

In some applications, the one or more rods include a rotation rod that is configured to rotate the cannulated screw.

In some applications, the one or more rods further include a pushing rod configured to push the cannulated screw, while it is being rotated.

In some applications, the one or more spools are coupled to the pushing rod.

There is further provided, in accordance with some applications of the present invention, a method including:
providing a suture anchor configured to be inserted into a hole in the bone, the suture anchor including:
   a tip component configured to be advanced into the hole, the tip component including a widened end portion that defines an eyelet that is configured to receive a suture, and a shaft that projects axially from the widened end portion, and
   a cannulated screw configured to secure the tip component within the hole within the bone by being advanced over the shaft of the tip component;
providing a suture-anchor insertion device that includes a handle portion, one or more rods, and one or more spools;
inserting a suture through the eyelet; and
releasably securing proximal ends of the suture by winding the proximal ends of the suture around the one or more spools.

There is further provided, in accordance with some applications of the present invention, apparatus for securing a suture to a bone, the apparatus including:
a suture anchor configured to be inserted into a hole in the bone, the suture anchor including:
   a tip component configured to be advanced into the hole, the tip component including:
      a widened end portion that defines an eyelet that is configured to receive the suture;
      a shaft that projects axially from the widened end portion; and
   a cannulated screw configured to secure the tip component within the hole within the bone by being advanced over the shaft of the tip component; and
a suture-anchor insertion device including:
   a handle portion;
   one or more rods; and
   one or more shoulder cleats extending substantially laterally at a distal end of the handle portion, the shoulder cleats being configured to releasably secure proximal ends of the suture by the proximal ends of the suture being inserted into the one or more shoulder cleats.

In some applications, the one or more shoulder cleats are shaped to define a proximally-facing concave curvature.

In some applications, the one or more shoulder cleats are configured to secure the proximal ends of the suture, such as to prevent twisting and/or knotting of the suture.

In some applications, the one or more shoulder cleats are configured to secure the proximal ends of the suture, such as to hold the proximal ends of the suture out of the way of the suture-anchor insertion device.

In some applications, the apparatus includes the suture, and the suture anchor insertion device is provided to a user with the suture passing through the eyelet and with proximal ends of the suture secured by the one or more shoulder cleats.

In some applications, the suture anchor insertion device is provided to a user without the suture passing through the eyelet and the apparatus is configured for a user to insert the suture through the eyelet and to insert the proximal ends of the suture into the one or more shoulder cleats.

In some applications, the one or more shoulder cleats are configured such that the proximal ends of the sutures are configured to be released from the one or more shoulder cleats prior to advancing the suture anchor into the hole in the bone.

In some applications, the one or more shoulder cleats are configured such that the proximal ends of the sutures are configured to be released from the one or more shoulder cleats subsequent to advancing the suture anchor into the hole in the bone.

In some applications, the one or more shoulder cleats are configured to allow a user to adjust tension of the suture.

In some applications, the one or more shoulder cleats include a trapping mechanism configured to mechanically trap the proximal ends of the suture such that the proximal ends of the suture cannot be pulled out of the shoulder cleats longitudinally but can be removed from the shoulder cleats by pulling the proximal ends of the suture laterally.

In some applications, the suture anchor defines a stay suture eyelet, and the apparatus further includes:
a stay suture configured to pass through the stay suture eyelet and into the handle portion, by passing along a lumen defined by one of the one or more rods; and
a stay-suture release tab configured to releasably hold a proximal end of the stay suture within the handle portion.

In some applications, the shoulder cleats extend laterally, such as to define lateral extensions that extend laterally past lateral edges of the handle portion.

In some applications, the lateral extensions of the shoulder cleats are configured to allow a user to adjust tension of the suture by pulling the proximal ends of the suture around the lateral extensions.

In some applications, the shoulder cleats define a trapping mechanism and are configured to allow the user to secure the suture at a desired tightness by sliding the proximal ends of the suture laterally from the lateral extensions and into the trapping mechanism.

In some applications, the handle portion includes a rotation knob that is configured to rotate the cannulated screw.

In some applications, the handle portion further includes a pusher disposed distally with respect to the rotation knob and configured to push the cannulated screw, while the cannulated screw is being rotated.

In some applications, the one or more shoulder cleats extend substantially laterally at a distal end of the pusher.

There is further provided, in accordance with some applications of the present invention, a method including:
providing a suture anchor configured to be inserted into a hole in the bone, the suture anchor including:
   a tip component configured to be advanced into the hole, the tip component including a widened end portion that defines an eyelet that is configured to receive a suture, and a shaft that projects axially from the widened end portion, and
   a cannulated screw configured to secure the tip component within the hole within the bone by being advanced over the shaft of the tip component;
providing a suture-anchor insertion device that includes a handle portion, one or more rods, and one or more shoulder cleats that extend substantially laterally at a distal end of the handle portion;
inserting a suture through the eyelet; and
releasably securing proximal ends of the suture by inserting the proximal ends of the suture into the one or more shoulder cleats.

There is further provided, in accordance with some applications of the present invention, apparatus for securing a suture to a bone, the apparatus including:
a suture anchor configured to be inserted into a hole in the bone, the suture anchor including:
   a tip component configured to be advanced into the hole, the tip component including:
      a widened end portion that defines an eyelet that is configured to receive the suture; and
      a shaft that projects axially from the widened end portion,
      the tip component defining a stay suture eyelet; and
   a cannulated screw configured to secure the tip component within the hole within the bone by being advanced over the shaft of the tip component; and
a suture-anchor insertion device including:
   a handle portion;
   one or more rods;
   a stay suture configured to pass through the stay suture eyelet and into the handle portion, by passing along a lumen defined by one of the one or more rods; and
   a stay-suture release tab configured to releasably hold a proximal end of the stay suture within the handle portion.

In some applications, the stay-suture release tab includes a tearable material and is configured to be manually torn by a user such as to release the proximal end of the stay suture.

In some applications, the stay-suture release tab includes a non-tearable material and is configured to be manually pulled by a user such as to release the proximal end of the stay suture.

In some applications, the apparatus is configured such that no portion of the stay suture is externally exposed until the proximal end of the stay suture is released.

There is further provided, in accordance with some applications of the present invention, a method for securing a suture to a bone, the method including:
inserting a tip component of a suture anchor into a hole in the bone, the tip component including a widened end portion that defines an eyelet through which the suture is placed, and a shaft that projects axially from the widened end portion,
the tip component further defining a stay suture eyelet;
advancing a cannulated screw over the shaft of the tip component such as to secure the tip component within the hole within the bone by pushing and rotating the cannulated screw using a suture-anchor insertion device that includes a handle portion and one or more rods,
a stay suture passes through the stay suture eyelet and into the handle portion, by passing along a lumen defined by one of the one or more rods, and a proximal end of the stay suture is releasably held within the handle portion by a stay-suture release tab;
the method further including, subsequent to advancing the cannulated screw over the shaft of the tip component releasing the proximal end of the stay suture from the handle portion by pulling and/or tearing the stay-suture release tab.

The present invention will be more fully understood from the following detailed description of applications thereof, taken together with the drawings, in which:

### BRIEF DESCRIPTION OF THE DRAWINGS

FIGS. 1A and 1B are schematic illustrations of a suture-anchor insertion device, in accordance with some applications of the present invention;

FIGS. 2A and 2B are schematic illustrations of the suture-anchor insertion device, respectively, without and with tissue-affixing sutures loaded thereon, in accordance with some applications of the present invention; and

FIGS. 3A, 3B, 3C, 3D, 3E, 3F, 3G, 3H, and 3I are schematic illustrations of respective steps of a procedure for inserting a suture anchor into a subject's bone using a suture-anchor insertion device, in accordance with some applications of the present invention.

### DETAILED DESCRIPTION OF EMBODIMENTS

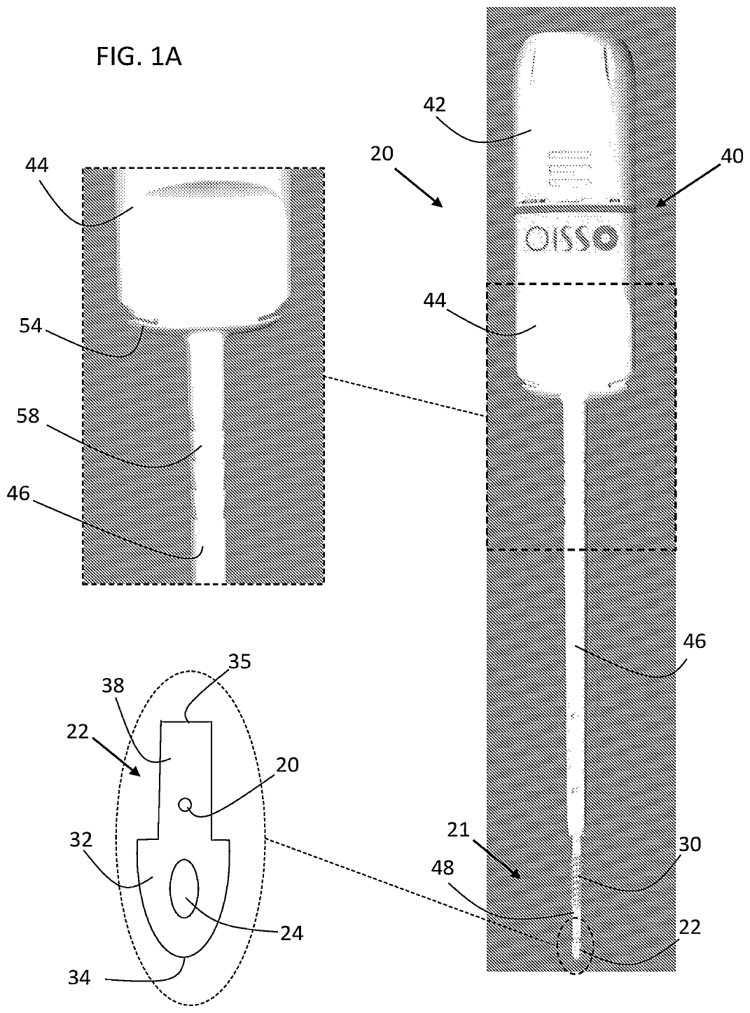

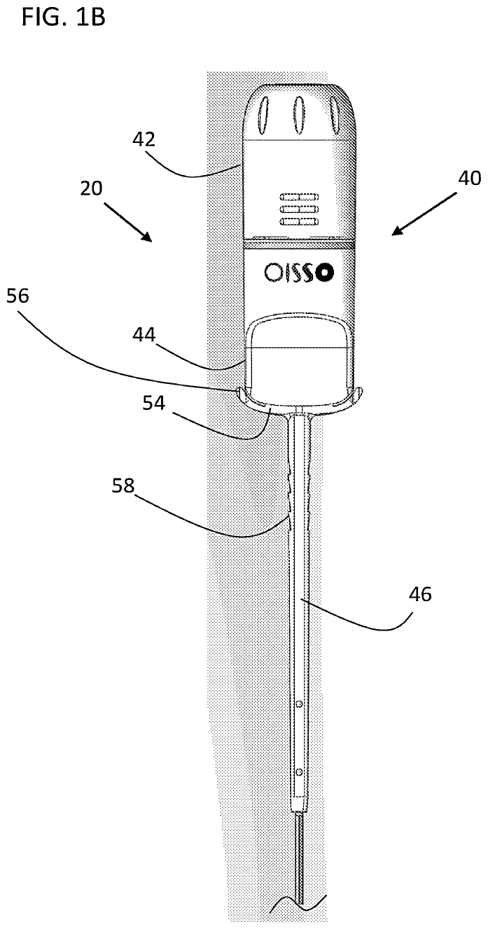

Reference is now made to FIGS. 1A and 1B, which are schematic illustrations of a suture-anchor insertion device 20, in accordance with some applications of the present invention. FIGS. 1A and 1B show respective examples of the suture-anchor insertion device, with the differences between these examples being described in further detail hereinbelow. In general, all aspects of the suture-anchor insertion devices shown in FIGS. 1A and 1B are similar to each other, except for the differences described herein.

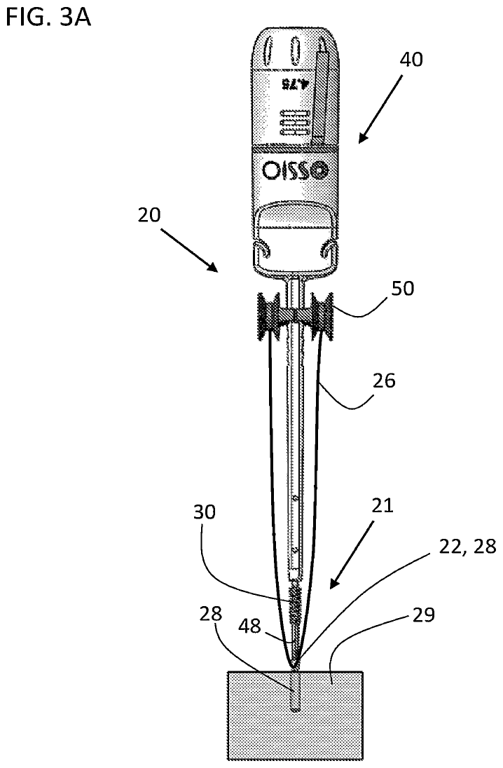

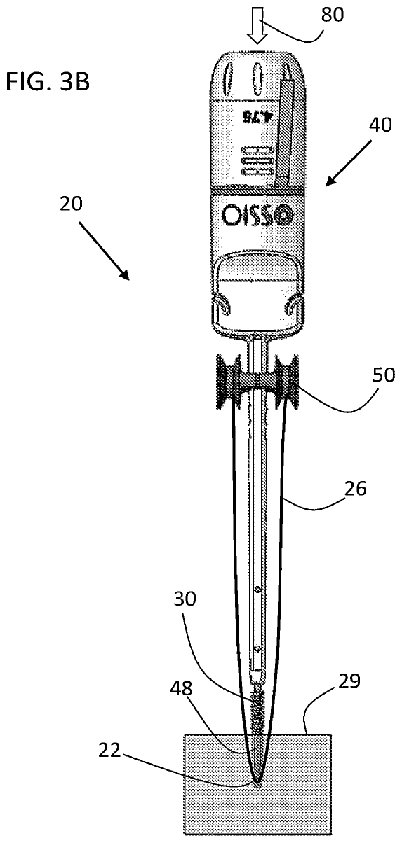

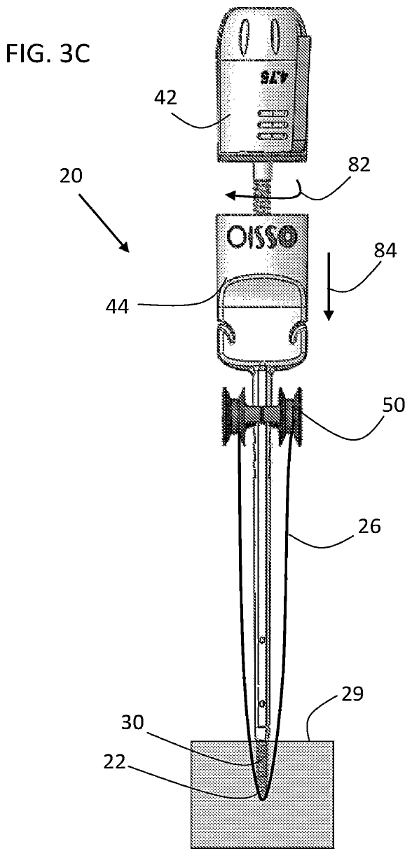

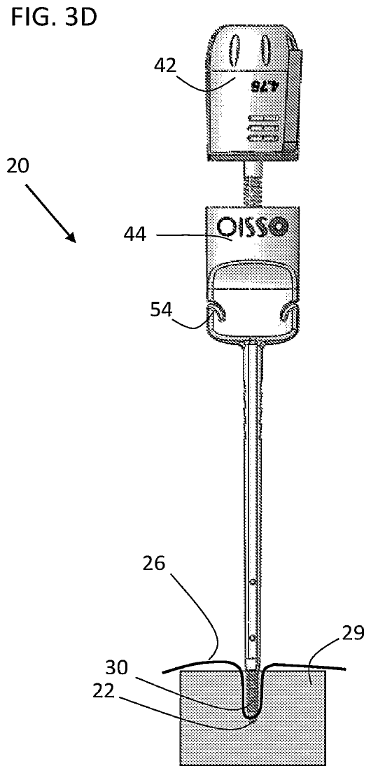

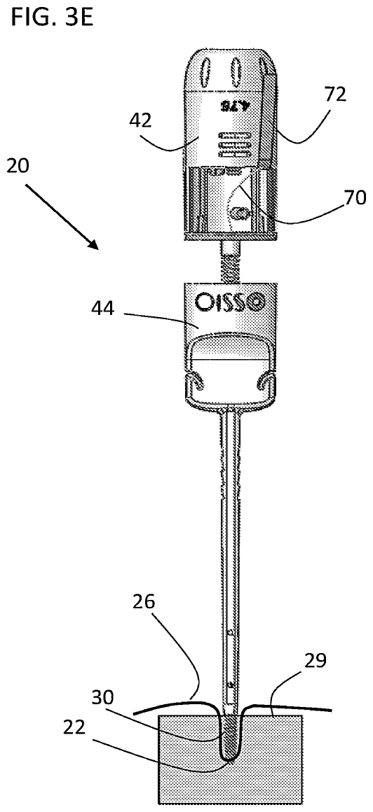

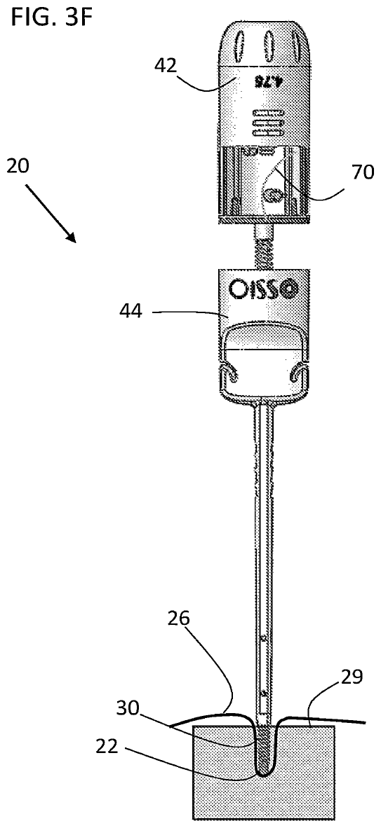

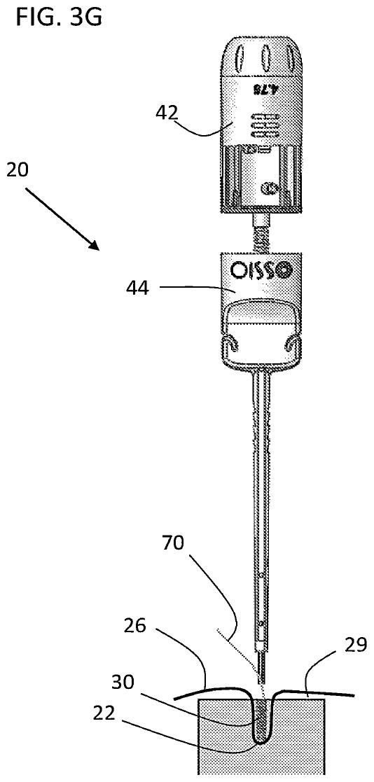

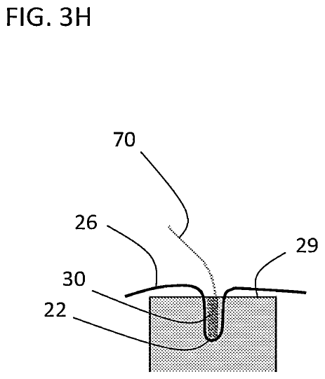

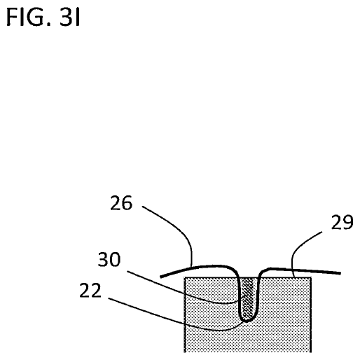

For some applications, suture-anchor insertion device 20 is used to insert a suture anchor 21 into bone, such that the suture anchor secures soft tissue to the bone in a manner that does not require the surgeon to tie suture knots to secure the tissue to the bone. Typically, suture anchor 21 includes a tip component 22 that defines an eyelet 24. The tip component is configured to hold one or more sutures (e.g., between one and four sutures), which are typically tissue-affixing sutures, within a pilot hole 28 in bone 29 (suture 26, pilot hole 28, and bone 29 all shown in FIGS. 3A-G). For some applications, some or all of the tissue-affixing sutures are attached to a curved or straight needle at one end or at both ends. The tissue-affixing sutures are typically threaded through one or more tendons or ligaments and are used to approximate the tendons or ligaments to the bone surface and to affix the tendons or ligaments to the bone.

Typically, the eyelet is a closed aperture and the tissue-affixing sutures are suture strands. For some applications (not shown), the eyelet is not a closed aperture. For example, the eyelet may define a portion of a circle, a portion of an ellipse, and/or a forked shape that is configured to hole the suture within the pilot hole. For some applications, tip component 22 defines a widened end portion 32 toward a first (distal) end 34 of the tip component, which is configured to be advanced to the bottom of the pilot hole. The widened end portion typically defines eyelet 24, through which the sutures are threaded. Typically, the tip component includes a shaft 38, which extends axially from the widened end portion of the tip component toward a second (proximal) end 35 of the tip component (which is configured to be placed closer to the top of the pilot hole).

For some applications, the suture anchor includes a plug component, which is configured to secure the tip component within the pilot hole by being advanced into and thereby plugging the pilot hole. Typically, the plug component is a cannulated screw 30, which is configured to secure the tip component within the pilot hole by being screwed into the pilot hole to thereby plug the pilot hole. Cannulated screw 30 typically defines a lumen therethrough, and the cannulated screw is typically advanced over shaft 38 of the tip component, such that the shaft is disposed within the lumen of the cannulated screw.

Suture-anchor insertion device 20 typically includes a handle portion 40 that includes a proximal rotation knob 42 and a distal pusher 44. (The term “proximal” is used herein to denote a portion that is closer to a user of the device (e.g., an orthopedic surgeon), while the term “distal” is used to denote a portion that is farther from the user.) Typically, a pushing rod 46 extends distally from the distal pusher to the proximal end of the cannulated screw. Further typically, a rotation rod 48 extends distally from the proximal rotation knob to the cannulated screw and through the lumen of the cannulated screw. For some applications, the rotation rod extends through the inside of the pushing rod, as shown. For some applications, the cannulated screw is advanced into the pilot hole and over shaft 38 of tip component 22 by using the pushing rod to push the screw distally, while simultaneously using the rotation rod to rotate the screw, and thereby screw the screw into the bone.

Typically, the rotation rod has a cross-sectional shape that conforms with an inner cross-sectional shape the defines the lumen of the cannulated screw, such that the rotation rod engages the internal surfaces of the cannulated screw. For example, the rotation rod may have a polygonal (e.g., a hexagonal shape) and the inner cross-sectional shape that defines the lumen of the cannulated screw may also be polygonal (e.g., hexagonal, as shown). Alternatively, other shapes may be used. Further alternatively or additionally, other methods may be used for rotating the canulated interference screw.

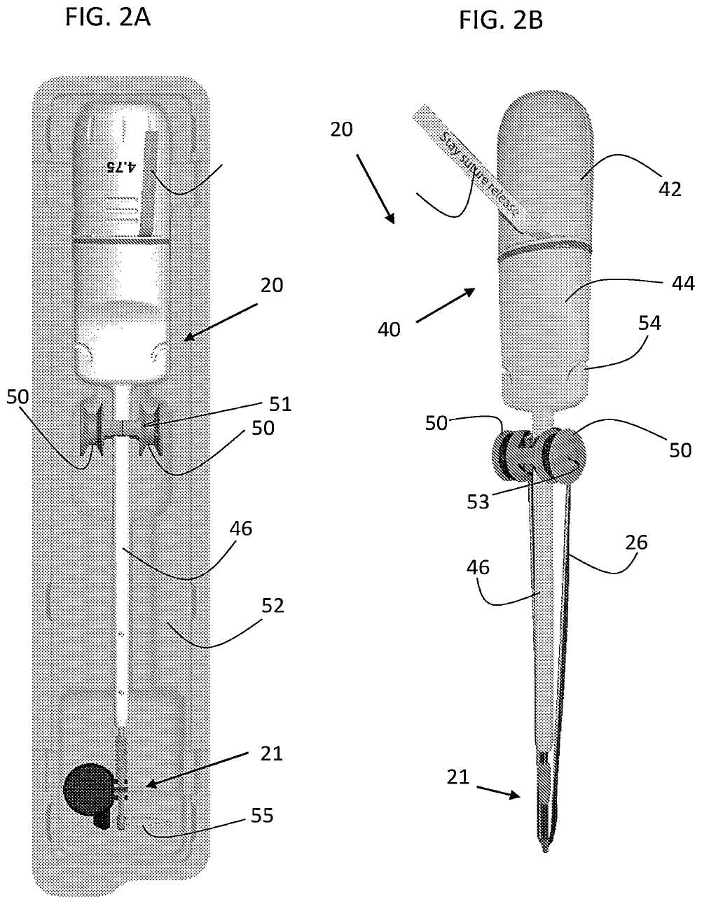

Reference is now made to FIG. 2A, which is a schematic illustrations of suture-anchor insertion device 20 as it is packaged in accordance with some applications of the present invention. Reference is also made to FIG. 2B, which is a schematic illustration of suture-anchor insertion device 20 with proximal ends of tissue-affixing suture(s) 26 being secured by spools 50, in accordance with some applications of the present invention. For some applications, suture-anchor insertion device 20 is provided to a user without the tissue-affixing suture(s) having been preloaded (e.g., as shown in FIG. 2A, in which the suture-anchor insertion device is shown inside packaging 52 in the absence of tissue-affixing suture(s)). Typically, for such applications, the suture-anchor insertion device is provided with a clip 55 that passes through eyelet 24 of tip component 22, in order to facilitate insertion of tissue-affixing suture(s) via the eyelet. Alternatively, the suture-anchor insertion device 20 is provided to a user with tissue-affixing suture(s) having been preloaded. For some such applications, the tissue-affixing suture(s) is provided preloaded through eyelet 24 of tip component 22 and with proximal ends of tissue-affixing suture(s) 26 having been secured by being wound around spools 50, as shown in FIG. 2B. Typically, the proximal ends of the tissue-affixing suture(s) are wound around the spools, such as to prevent damage to the tissue-affixing suture(s) (e.g., twisting and/or knotting of the tissue-affixing suture(s)) while the tissue-affixing suture(s) are disposed within the packaging. Typically, the spools secure the proximal ends of the tissue-affixing suture(s) with respect to pushing rod 46 and/or with respect to handle portion 40.

For some applications, suture-anchor insertion device 20 is provided to a user without the tissue-affixing suture(s) having been preloaded, but the insertion device is provided with spools Typically, once the tissue-affixing suture(s) has been inserted through eyelet 24 of tip component 22 by the user, the user then winds the proximal ends of the tissue-affixing suture(s) around spools 50. Thus, irrespective of whether or not the insertion device is provided with the tissue-affixing suture(s) having been inserted through eyelet 24 of tip component 22, for some applications, prior to insertion of the suture anchor and/or during the insertion of the suture anchor, the proximal ends of the tissue-affixing suture(s) are wound around the spools. Typically, the proximal ends of the tissue-affixing suture(s) are wound around the spools, such as to prevent damage to the tissue-affixing suture(s) (e.g., twisting and/or knotting of the tissue-affixing suture(s)) and to hold the proximal ends of the tissue-affixing suture(s) out of the way of the insertion device. For some applications, prior to insertion of the suture anchor and/or during the insertion of the suture anchor, the user is able to adjust the tension of the tissue-affixing suture(s) by winding the spools. For some applications, suture-anchor insertion device 20 is provided to a user with the tissue-affixing suture(s) having been preloaded onto the spools, and the user removes the proximal ends of the tissue-affixing suture(s) from the spools prior to the insertion of the suture anchor.

As described hereinabove, for some applications, some or all of tissue-affixing sutures 26 are attached to a curved or straight needle at one end or at both ends. For some applications, spools 50 are configured to safely store the needles. For example, the spools may define holes 51 that are shaped so as to receive tips of the needles and to thereby hold the needles in a way that does not expose the sharp tips of the needles. For some applications, the spool is shaped such as to accommodate the needle, for example, the needles may be curved and the radius of curvature of spool is similar to that of the needles, such that the spool accommodates the curvature of the needle.

Typically, after suture anchor 21 has been inserted, tissue-affixing sutures 26 are released from the spools by pulling the tissue-affixing sutures 26 while allowing the spools to rotate and release the strands, for example by holding the sides of the spools lightly and allowing them to rotate. For some applications, the spools define round indents 53 on their outer surfaces such as to allow a user to insert their fingers into the indents and thereby hold the spools while they are rotated as the sutures are pulled off. As noted above, for some applications, the user removes the proximal ends of the tissue-affixing suture(s) from the spools prior to the insertion of the suture anchor.

Typically, spools 50 are detachably attached to the suture-anchor insertion device such that the spools are configured to remain attached while the suture anchor is being inserted but can be readily removed following suture anchor insertion. For some applications, the user removes the spools from the suture-anchor insertion device prior to the insertion of the suture anchor. For some applications, the spools are attached to a rod of the suture-anchor insertion device, e.g., to pushing rod 46 of the suture-anchor insertion device. For some such applications, pushing rod 46 includes a segment 58 having a non-smooth surface (e.g., a segment that defines teeth, waves, and/or a ribbed segment, shown in FIG. 1) in order to facilitate coupling of the spools to the pushing rod. Typically, the suture-anchor insertion device includes two spools (e.g., with one spool on each side of the suture anchor). For some such applications, the spools are coupled to each other. Typically, the spools are removable from the pushing rod with a single motion, for example by pulling the spools from the pushing rod. Typically, the spool is comprised of a material having the same or higher flexibility than the flexibility of the pushing rod.

Referring again to FIGS. 1A and 1B (which show respective examples of the suture-anchor insertion device in the absence of suture 26), for some applications, handle portion 40 of insertion device 20 defines shoulder cleats 54. For example, as shown, the distal end of the handle portion (i.e., the distal end of pusher 44) may define shoulder cleats. Typically, the shoulder cleats extend substantially laterally with respect to the handle portion. (It is noted that the shoulder cleats may also extend in additional directions, other than the lateral direction. For example, the shoulder cleats may be shaped to define a proximally-facing concave curvature, as shown.) For some applications, the shoulder cleats extend laterally to the edges of the handle portion, e.g., as shown in FIG. 1A. Alternatively, the shoulder cleats extend laterally past the lateral edges of the handle portion, e.g., as shown in FIG. 1B. For some applications, as an alternative to or in addition to winding the proximal ends of tissue-affixing suture(s) 26 around spools 50, the proximal ends of tissue-affixing suture(s) 26 are inserted into the shoulder cleats. For some such applications, the tissue-affixing suture(s) is provided preloaded through eyelet 24 of tip component 22 and with proximal ends of tissue-affixing suture(s) 26 having been secured (typically, with respect to the handle portion) by having been inserted into the shoulder cleats. Typically, the proximal ends of the tissue-affixing suture(s) are secured by the shoulder cleats, such as to prevent damage to the tissue-affixing suture(s) (e.g., twisting and/or knotting of the tissue-affixing suture(s)) while the tissue-affixing suture(s) are disposed within the packaging. For some applications, suture-anchor insertion device 20 is provided to a user without the tissue-affixing suture(s) having been preloaded, but the insertion device is provided with shoulder cleats 54. Typically, once the tissue-affixing suture(s) has been inserted through eyelet 24 of tip component 22 by the user, the user then inserts the proximal ends of the tissue-affixing suture(s) into shoulder cleats 54. Thus, irrespective of whether or not the insertion device is provided with the tissue-affixing suture(s) having been inserted through eyelet 24 of tip component 22, for some applications, prior to insertion of the suture anchor and/or during the insertion of the suture anchor, the proximal ends of the tissue-affixing suture(s) are inserted into the shoulder cleats. Typically, the proximal ends of the tissue-affixing suture(s) are inserted into the shoulder cleats, such as to prevent damage to the tissue-affixing suture(s) (e.g., twisting and/or knotting of the tissue-affixing suture(s)) and to hold the proximal ends of the tissue-affixing suture(s) out of the way of the insertion device. For some applications, suture-anchor insertion device 20 is provided to a user with the tissue-affixing suture(s) having been inserted into the shoulder cleats, and the user removes the proximal ends of the tissue-affixing suture(s) from the shoulder cleats prior to the insertion of the suture anchor.

Typically, the shoulder cleats include a trapping mechanism (e.g., a male-female interlocking structure) for mechanically trapping the proximal ends of the tissue-affixing suture(s) such that the proximal ends of the tissue-affixing suture(s) cannot be pulled out longitudinally but can be removed by pulling the proximal ends of the tissue-affixing suture(s) laterally. As described hereinabove, for some applications, the shoulder cleats extend laterally outwardly past the lateral edges of the handle portion, e.g., as shown in FIG. 1B. For some applications, suture-anchor insertion device 20 is provided to a user without the tissue-affixing suture(s) having been preloaded, but the insertion device is provided with shoulder cleats 54. Typically, once the tis sue-affixing suture(s) has been inserted through eyelet 24 of tip component 22 by the user, the user then inserts the proximal ends of the tissue-affixing suture(s) into shoulder cleats 54. For some such applications, lateral extensions 56 of the shoulder cleats (which extend laterally past the lateral edges of the handle portion), allow the user to initially adjust the tension of the tissue-affixing suture(s) by pulling proximal ends of the tissue-affixing suture(s) around the lateral extensions. Subsequently, the tissue-affixing suture(s) are secured at the desired tightness by sliding the proximal ends of the tissue-affixing suture(s) laterally into the trapping mechanism.

For some applications, the suture-anchor insertion device includes a stay suture 70 (shown in FIGS. 3E-G, for example), and a stay-suture release tab 72. The stay suture typically passes through a stay-suture eyelet 74 defined by shaft 38 of tip component 22 of the suture anchor, and then passes through a lumen defined by rotation rod 48 to handle portion 40 (e.g., to rotation knob 42). The stay suture is typically provided preloaded within the insertion device such that the tip component is fixed to the distal end of the rotation rod. At its proximal end, the stay suture is held in position with respect to the rotation knob via the stay-suture release tab. In order to release the tip component from the insertion device once the suture anchor has been properly implanted, the stay-suture release tab is typically manually removed (e.g., pulled away and/or ripped). This is described in further detail hereinbelow with reference to FIGS. 3E-H.

Typically, the suture-anchor insertion device is provided to the user with cannulated screw pre-mounted on rotation rod 48 above tip component 22, such that when the proximal end of the cannulated screw is in contact with the distal end of pushing rod 46, there is an axial gap between the cannulated screw and the tip component. For some applications, the length of the gap is approximately equal to the length of the pilot hole.

Reference is now made to FIGS. 3A, 3B, 3C, 3D, 3E, 3F, 3G, 3H, and 3I, which are schematic illustrations of respective steps of a procedure in which a suture 26 is secured to bone 29, in accordance with some applications of the present invention.

Typically, in a first step, pilot hole 28 is drilled into bone 29. FIG. 3A schematically illustrates bone 29 with pilot hole 28 having already been drilled, and with the suture-anchor insertion device prepared for insertion of the suture anchor into the pilot hole, with the tissue-fixation suture(s) inserted through eyelet 24 of tip component 22 and with the proximal ends of the tissue-fixation suture(s) wound around spools 50. (As described hereinabove, alternatively or additionally, the proximal ends of the tissue-fixation suture(s) wound around shoulder cleats 54.) For some applications, pilot hole 28 is equal to the total length of the suture anchor 21. Alternatively, the pilot hole is shorter than the total length of the suture anchor 21, e.g., up to half of the total length of the suture anchor. Cannulated screw 30 is mounted on rotation rod 48 above tip component 22, with an axial gap between the cannulated screw and the tip component. As described hereinabove, for some applications, the length of the gap is approximately equal to the length of the pilot hole.

Referring now to FIG. 3B, in a subsequent step, tip component 22 and rotation rod 48 are advanced into the pilot hole. For some applications, the tip component 22 and rotation rod 48 are advanced into the pilot hole by gently tapping an upper end of handle portion 40, as indicated by arrow 80. For some applications, the rotation rod includes a marking (e.g., a laser-cut marking) to indicate a depth to which to advance the rotation rod into the pilot hole. As noted above, for some applications, the pilot hole is shorter than the total length of the suture anchor 21, e.g., up to half of the total length of the suture anchor. For such applications, the tip component is advanced into bone 29 to a greater depth than the depth of the pilot hole that was drilled. Typically, this is possible because cancellous bone tissue is spongy. Further typically, this results in the tip component being more securely anchored in place within the pilot hole (and the suture thereby being more securely anchored within the pilot hole) relative to the tip element is advanced into bone 29 only as far as the depth of the pilot hole that was drilled. Typically, the rotation rod 48 is advanced into the pilot hole, such that the distal end of cannulated screw 30 is disposed at the entrance to the pilot hole.

Referring now to FIG. 3C, typically, the cannulated screw is screwed into the pilot hole by simultaneously (a) rotating rotation rod 48 (as indicated by arrow 82), and (b) pushing cannulated screw 30 axially along rotation rod 48 using pushing rod 46 (as indicated by arrow 84).

Referring to FIG. 3D, once the cannulated screw has been fully screwed into the pilot hole (and typically, once the distal end of the screw is fully advanced over shaft 38 of tip component 22, such that the widened end portion of the tip component blocks the screw from advancing further with respect to the tip component), the proximal ends of the tissue-fixation suture(s) are released. Typically, after suture anchor 21 has been inserted, tissue-affixing sutures 26 are released from the spools by pulling the tissue-affixing sutures 26 while allowing the spools to rotate and release the sutures, for example by holding the sides of the spools lightly and allowing them to rotate. As described hereinabove, for some applications, the spools define round indents 53 (shown in FIG. 2B) on their outer surfaces such as to allow a user to insert their fingers into the indents and thereby hold the spools while they are rotated as the sutures are pulled off. As noted above, for some applications, the user removes the proximal ends of the tissue-affixing suture(s) from the spools prior to the insertion of the suture anchor.

As described hereinabove, typically, spools 50 are detachably attached to the suture-anchor insertion device such that the spools are configured to remain attached while the suture anchor is being inserted but can be readily removed following suture anchor insertion. For some applications, the user removes the spools from the suture-anchor insertion device prior to the insertion of the suture anchor. Typically, the suture-anchor insertion device includes two spools, with one on each side of the suture anchor. For some such applications, the spools are coupled to each other. Typically, the spools are removable from the pushing rod with a single motion, for example by pulling the spools from the pushing rod. Typically, the spool is comprised of a material having the same or higher flexibility than the flexibility of the pushing rod.

For applications in which the proximal ends of the tissue-affixing suture(s) are inserted into shoulder cleats 54, the proximal ends of the tissue-affixing suture(s) are typically removed from the shoulder cleats at this stage. As described above, typically, the shoulder cleats include a trapping mechanism (e.g., a male-female interlocking mechanism) for mechanically trapping the tissue-affixing suture(s) such that the tissue-affixing suture(s) cannot be pulled out longitudinally but can be removed by pulling the tissue-affixing suture(s) to the side. For such applications, the tissue-affixing suture(s) are released from the mechanism in order to remove them from the shoulder cleats. For some applications, the user removes the proximal ends of the tissue-affixing suture(s) from the shoulder cleats prior to the insertion of the suture anchor.

Referring to FIG. 3E, as described hereinabove, for some applications, the suture-anchor insertion device includes stay suture 70 and stay-suture release tab 72. The stay suture typically passes through a stay-suture eyelet 74 defined by shaft 38 of tip component 22 of the suture anchor, and then passes through a lumen defined by rotation rod 48 to handle portion 40 (e.g., to rotation knob 42). The stay suture is typically provided preloaded within the insertion device such that the tip component is fixed to the distal end of the rotation rod. At its proximal end, the stay suture is held in position with respect to the rotation knob via the stay-suture release tab (e.g., by the stay-suture release tab adhering the stay suture to an inner surface of the rotation knob). FIG. 3E shows the rotation knob with a portion of its cover removed, such that coupling between the stay suture and the stay-suture release tab is visible.

In order to release the tip component from the insertion device once the suture anchor has been properly implanted, the stay-suture release tab is typically manually removed (e.g. pulled away and/or ripped). For some applications, the stay-suture release tab is made of a manually tearable material, such as paper or fabric, that is configured to be manually torn such as to release the stay suture. Alternatively, the stay-suture release tab is made of is made of a non-tearable material (e.g., a material made of high-density spunbound polyethylene fibers, such as Tvvek®) that is configured to be pulled away such as to release the stay suture. In an alternative embodiment (not shown), a mechanical element such as a button or a lever is used to release the stay suture. Further alternatively, the stay suture may be released by opening up the handle portion (e.g., by opening up rotation knob 42 of the handle portion).

FIG. 3F shows the suture-anchor insertion device with the stay-suture release tab having been removed, such that the stay suture is released. Typically, the release of the stay suture decouples the tip component from the distal end of the rotation rod, thereby allowing the rotation rod to be retracted from inside the cannulated screw, as shown in FIGS. 3G and 3H. Typically, the stay suture is then simply pulled out of the stay suture eyelet, leaving only the suture anchor (including tissue-affixing suture(s) 26) in placed in the bone, as shown in FIG. 3I. As described hereinabove, the tissue-affixing suture(s) are typically threaded through one or more tendons or ligaments, with the tissue-affixing suture(s) being used to approximate the tendons or ligaments to the bone surface to the affix tendons or ligaments to the bone.

It is noted that an alternative configuration from that described with reference to FIGS. 3E-H is for the stay suture to pass from the eyelet of the tip component all the way through an internal cannula of the suture-anchor insertion device and out of the proximal end of the suture-anchor insertion device. For such applications, the stay suture is typically fixated to the suture-anchor insertion device, for example by winding the suture around a crevice at the back of the suture-anchor insertion device. However, in such applications, releasing the stay suture typically requires unwinding of the stay suture from the device, which is burdensome. Using the configuration that is shown in FIGS. 3E-H, the stay suture only passes through the rotation rod, which is substantially shorter than the entire length of the insertion device. Furthermore, the stay suture is protected by the rotation rod and the handle portion, such that no portion of the stay suture is externally exposed until the stay suture is released.

It is noted that although some applications of the present disclosure are described as being applied to tissue-affixing suture(s), the scope of the present disclosure includes applying the apparatus and methods described herein to one or more sutures of any type, mutatis mutandis.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for securing a suture to a bone, the apparatus comprising:
a suture anchor configured to be inserted into a hole in the bone, the suture anchor comprising:
a tip component configured to be advanced into the hole, the tip component comprising:
a widened end portion that defines an eyelet that is configured to receive the suture;
a shaft that projects axially from the widened end portion; and
a cannulated screw configured to secure the tip component within the hole within the bone by being advanced over the shaft of the tip component; and
a suture-anchor insertion device comprising:
a handle portion;
one or more rods; and
a, one or more spools configured to releasably secure proximal ends of the suture by the proximal ends of the suture being wound around the one or more spools; or
b, one or more shoulder cleats extending substantially laterally at a distal end of the handle portion, the shoulder cleats being configured to releasably secure proximal ends of the suture by the proximal ends of the suture being inserted into the one or more shoulder cleats.

2. The apparatus according to claim 1, wherein the one or more spools are configured to secure the proximal ends of the suture, such as to hold the proximal ends of the suture out of the way of the suture-anchor insertion device and as to prevent twisting and/or knotting of the suture.

3. (canceled)

4. The apparatus according to claim 1, wherein the apparatus comprises the suture, and wherein the suture anchor insertion device is provided to a user with the suture passing through the eyelet and with proximal ends of the suture wound around the one or more spools.

5.-7. (canceled)

8. The apparatus according to claim 1, wherein the one or more spools are configured to allow a user to adjust tension of the suture by winding the one or more spools.

9. The apparatus according to claim 1, wherein the suture anchor defines a stay suture eyelet, the apparatus further comprising:
a stay suture configured to pass through the stay suture eyelet and into the handle portion, by passing along a lumen defined by one of the one or more rods; and
a stay-suture release tab configured to releasably hold a proximal end of the stay suture within the handle portion.

10. The apparatus according to claim 1, wherein the apparatus is configured for use with a suture that is coupled to a needle, and wherein the one or more spools are configured to accommodate the needle.

11. The apparatus according to claim 10, wherein the apparatus is configured for use with a suture that is coupled to a curved needle, and wherein the one or more spools has a curvature that is such as to accommodate a curvature of the curved needle; and wherein the one or more spools define one or more holes that are configured to receive a tip of the needle.

12. (canceled)

13. The apparatus according to claim 1, wherein the one or more spools are detachably coupled to the one or more rods.

14.-15. (canceled)

16. The apparatus according to claim 13, wherein the one or more rods include a segment having a non-smooth surface to facilitate coupling of the one or more spools to the one or more rods.

17. The apparatus according to claim 13, wherein the one or more spools comprises two or more spools that are coupled to each other, such as to be detached from the one or more rods simultaneously with each other.

18. (canceled)

19. The apparatus according to claim 1, wherein one or more spools are configured such that the suture is released from the spools by pulling the sutures while allowing the spools to rotate and release the suture.

20. The apparatus according to claim 19, wherein the one or more spools define round indents on outer surfaces thereof, which are configured such as to allow a user to insert their fingers into the indents and thereby hold the spools while the spools are rotated.

21. The apparatus according to claim 1, wherein the one or more rods comprise a rotation rod that is configured to rotate the cannulated screw.

22. The apparatus according to claim 21, wherein the one or more rods further comprise a pushing rod configured to push the cannulated screw, while it is being rotated.

23. The apparatus according to claim 22, wherein the one or more spools are coupled to the pushing rod.

24.-26. (canceled)

27. The apparatus according to claim 1, wherein the one or more shoulder cleats are configured to secure the proximal ends of the suture, such as to hold the proximal ends of the suture out of the way of the suture-anchor insertion device and prevent twisting and/or knotting of the suture.

28.-29. (canceled)

30. The apparatus according to claim 1, wherein the suture anchor insertion device is provided to a user without the suture passing through the eyelet and wherein the apparatus is configured for a user to insert the suture through the eyelet and to insert the proximal ends of the suture into the one or more shoulder cleats.

31.-33. (canceled)

34. The apparatus according to claim 1, wherein the one or more shoulder cleats comprise a trapping mechanism configured to mechanically trap the proximal ends of the suture such that the proximal ends of the suture cannot be pulled out of the shoulder cleats longitudinally but can be removed from the shoulder cleats by pulling the proximal ends of the suture laterally.

35. (canceled)

36. The apparatus according to claim 1, wherein the shoulder cleats extend laterally, such as to define lateral extensions that extend laterally past lateral edges of the handle portion.

37. (canceled)

38. The apparatus according to claim 1, wherein the shoulder cleats define a trapping mechanism and are configured to allow the user to secure the suture at a desired tightness by sliding the proximal ends of the suture laterally from the lateral extensions and into the trapping mechanism.

39.-47. (canceled)
